(19) United States
(12) Patent Application Publication
(10) Pub. No.: US 2025/0057631 A1
(43) Pub. Date: Feb. 20, 2025

(54) BIOPSY SITE MARKER WITH EXPANDABLE MESH

(71) Applicant: Devicor Medical Products, Inc., Cincinnati, OH (US)

(72) Inventors: James O. ROGERS, III, Cincinnati, OH (US); Jack A. RANDALL, Cincinnati, OH (US)

(21) Appl. No.: 18/934,643

(22) Filed: Nov. 1, 2024

Related U.S. Application Data
(63) Continuation of application No. PCT/US2023/022467, filed on May 17, 2023.
(60) Provisional application No. 63/354,952, filed on Jun. 23, 2022.

(51) Int. Cl. A61B 90/00

(52) U.S. Cl. CPC A61B 90/39

(57) ABSTRACT

A biopsy site marker includes a carrier and a marker element. The carrier is configured to expand in diameter in response to moisture. The marker element includes a base portion. The base portion defines a plurality of interconnected legs. Each leg is configured to move relative to another leg to permit movement of the marker element from a pre-deployment configuration to a post-deployment configuration. The base portion is disposed around an outer surface of the carrier. The carrier is configured to drive movement of the marker element from the pre-deployment configuration to the post-deployment configuration.

### PRIORITY

This application claims priority to U.S. Provisional Application Ser. No. 63/354,952, entitled “Biopsy Site Marker with Expandable Mesh,” filed on Jun. 23, 2022, the disclosure of which is incorporated by reference herein.

### BACKGROUND

Breast biopsies may be performed because of irregular mammograms and palpable abnormalities. Biopsies can include surgical excisional biopsies and stereotactic and ultrasound guided needle breast biopsies. In the case of image directed biopsy, the radiologist or other physician may take a small sample of the irregular tissue for laboratory analysis. If the biopsy proves to be malignant, additional surgery (e.g., a lumpectomy or a mastectomy) may be required. In the case of needle biopsies, the patient may return to the radiologist a day or more later, and the biopsy site (the site of the lesion) may need to be relocated in preparation for the surgery. An imaging system, such as ultrasound, magnetic resonance imaging (MRI) or x-ray may be used to locate the biopsy site. In order to assist the relocation of the biopsy site, a marker may be placed at the time of the biopsy.

The use of markers used after breast biopsies to mark the location where the biopsied tissue was removed is described in the following US patents: U.S. Pat. No. 6,083,524, titled “Polymerizable biodegradable polymers including carbonate or dioxanone linkages,” issued Jul. 4, 2000; U.S. Pat. No. 6,162,241, titled “Hemostatic tissue sealants,” issued Dec. 4, 2000; U.S. Pat. No. 6,270,464, titled “Biopsy localization method and device,” issued Aug. 7, 2001; U.S. Pat. No. 6,356,782, titled “Subcutaneous cavity marking device and method,” issued Mar. 12, 2002; U.S. Pat. No. 6,605,294, titled “Methods of using in situ hydration of hydrogel articles for sealing or augmentation of tissue or vessels,” issued Aug. 12, 2003; U.S. Pat. No. 8,600,481, titled “Subcutaneous cavity marking device,” issued Dec. 3, 2013 and U.S. Pat. No. 8,939,910, titled “Method for enhancing ultrasound visibility of hyperechoic materials”, issued Jan. 27, 2015. All of these US patents are incorporated by reference in their entirety.

Once a marker is placed at a biopsy site, the marker can later be relocated to identify the biopsy site in subsequent follow-up procedures. In some contexts, a placed marker may not completely correspond to the biopsy site when the marker is relocated. For instance, the marker may migrate to another nearby location during the intervening time between the biopsy procedure and subsequent follow up procedures. Migration of the biopsy site marker may cause difficulties when identifying the biopsy site during subsequent follow-up procedures. Accordingly, it may be desirable to incorporate features into a marker to maintain the marker in a fixed position over time.

While several systems and methods have been made and used for marking a biopsy site, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an example of a biopsy site marker;

FIG. 2 depicts a perspective view of an example of a marker delivery device for use with the marker of FIG. 1;

FIG. 3A depicts a perspective view of the marker of FIG. 1 being inserted into tissue using the marker delivery device of FIG. 2, the marker being in a pre-deployment configuration;

FIG. 3B depicts another perspective view of the marker of FIG. 1 being inserted into tissue using the marker delivery device of FIG. 2, the marker being in an expanded configuration;

FIG. 3C depicts yet another perspective view of the marker of FIG. 1 being inserted into tissue, the marker being in a post-deployment configuration;

FIG. 4 depicts a perspective view of another example of a biopsy site marker;

FIG. 5A depicts a perspective view of the marker of FIG. 4 being inserted into tissue using the marker delivery device of FIG. 2, the marker being in a pre-deployment configuration;

FIG. 5B depicts another perspective view of the marker of FIG. 4 being inserted into tissue using the marker delivery device of FIG. 2, the marker being in an expanded configuration;

FIG. 6 depicts a perspective view of yet another example of a biopsy site marker;

FIG. 7A depicts a perspective view of the marker of FIG. 6 being inserted into tissue using the marker delivery device of FIG. 2, the marker being in a pre-deployment configuration; and

FIG. 7B depicts another perspective view of the marker of FIG. 6 being inserted into tissue using the marker delivery device of FIG. 2, the marker being in an expanded configuration.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It may be beneficial to be able to mark the location or margins of a lesion, whether temporarily or permanently, prior to or immediately after removing or sampling it. Marking prior to removal may help to ensure that the entire lesion is excised, if desired. Alternatively, if the lesion were inadvertently removed in its entirety, marking the biopsy site immediately after the procedure would enable reestablishment of its location for future identification.

Once a marker is positioned at a biopsy site, it may be desirable for the marker to remain visible under ultrasound. It may also be desirable to make the marker readily identifiable relative to other structural features of a patient. For instance, it may be desirable for the marker to be distinguishable under ultrasound visualization from microcalcifications to avoid inadvertently characterizing the marker as a microcalcification during subsequent ultrasonic examinations. Generally, microcalcifications are used in the field to identify suspicious lesions or masses. Thus, it is generally desirable for the ultrasound view to be distinguishable as a marker and not inadvertently identified as a new mass.

### I. EXAMPLE SYSTEM FOR MARKER WITH LIMITED MIGRATION

In some versions of a biopsy site marker, it may be desirable to include certain features within the marker, or within portions of the marker, to reduce the propensity of the marker to migrate when placed within tissue. For instance, some markers may be prone to migration after placement at a biopsy site due to movement of tissue in the intervening time between marker placement and subsequent follow-up procedures. In addition or in the alternative, structures within tissue may increase the propensity of a marker to migrate. One example of such structures may include, for example, the passageway formed to for collection of a biopsy sample and/or placement of a marker. As a result, such markers and/or the surrounding environment may introduce challenges with accurately identifying the biopsy site during subsequent follow-up procedures. Accordingly, it may be desirable to incorporate features into a marker to assist in maintaining the marker in a generally fixed position within tissue over time. Although several versions are described herein that incorporate the features described above, it should be understood that various alternative combinations can be used without departing from the basic principles described herein.

A. Example of Biopsy Site Marker with Expandable Mesh

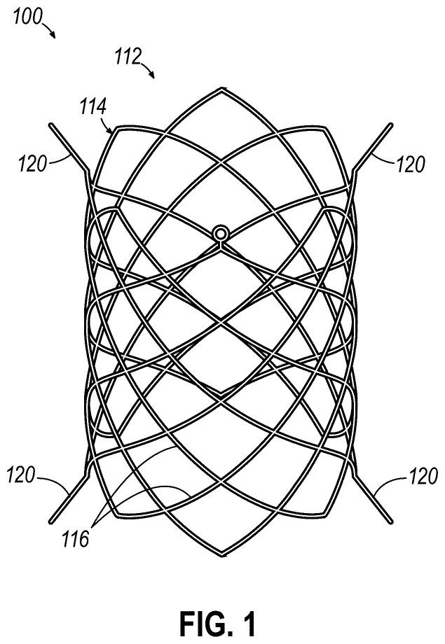

FIG. 1 shows an illustrative marker (100) that is generally configured to expand from a pre-deployment configuration to a post-deployment configuration to thereby anchor or otherwise fix marker (100) within tissue at a predetermined location. Marker (100) of the present version includes a marker element (112). In some versions, marker element (112) may be generally configured as non-bioabsorbable and radiopaque and/or echogenic to enhance visualization using one or more modes of visualization.

Additionally, although not shown, it should be understood that in some versions, marker (100) may include structures that may be referred to as a “carrier.” By way of example only, such carriers may include certain bioabsorbable features that may be absorbed into tissue over time. In some versions, marker element (112) may be embedded within such carriers. In such versions, one or more portions of marker element (112) may protrude outwardly from such carriers. In other versions, marker element (112) may surround such carriers, with the carrier acting as a core. In versions including such carriers, carriers may be shaped as described in greater detail below or alternatively configured as a coating. In other versions, marker (100) may only include marker element (112), omitting additional structures like carriers. In other words, in some versions, marker (100) may be configured as a “bare” marker as shown in FIG. 1. It should be understood that in versions using elements such as a carrier, such carriers may be configured to provide enhanced visualization under certain imaging modalities. By way of example only, such carriers may include echogenic features to enhance visibility of marker (100) under ultrasound.

As best seen in FIG. 1, marker element (112) includes a body (114) and a plurality of anchors (120) extending outwardly from one or more portions of body (114). Body (114) is generally configured to readily expand and contract between different positions, as will be described in greater detail below. Body (114) in the present version includes a plurality of interconnecting legs (116). Legs (116) may be generally arranged to form a repeating pattern of one or more predetermined shapes. For instance, in the present version, legs (116) form a plurality of repeating triangular or zig-zag shapes. In other versions, a plurality of other alternative shapes may be used either alone or in combination. Examples of suitable shapes formed by legs (116) may include triangular, zig-zag, square, rectangular, oval-shaped, circular, octagonal, and/or etc. Additionally, although each leg is shown as being a generally straight section of wire, it should be understood that in other versions, each leg (116) may be curved to provide different irregular shapes.

Regardless of the specific shape formed by legs (116), it should be understood that legs (116) are generally configured to facilitate deformation of marker element (112) between one or more different positions. Thus, the particular shape formed by legs (116) is generally configured permit at least some movement of individual legs (116). Some flexion of individual legs (116) relative to other legs (116) may be desirable to permit deformation of marker element (112) generally and/or one or more portions of marker element (112). The particular shape formed by legs (116) may also be desirable to provide marker element (112) with a relatively large footprint and/or surface area using a relatively small volume of material. The particular shape formed by legs (116) may additionally be desirable to provide openings within the surface of marker element (112) to facilitate tissue in growth, thereby providing additional tissue anchoring and/or fixation over time.

Legs (116) are interconnected with each other to form a general shape of marker element (112). In the present version, marker element (112) is formed in the shape of a hollow cylinder (e.g., stent-shaped, either straight or tapered). As will be described in greater detail below, this particular shape may be desirable to facilitate deployment of marker (100) at a biopsy site. Although both ends of marker element (112) are open in the present version, in other versions, one or both ends may be closed. In still other versions, a variety of alternative shapes may be used. Suitable alternative shapes for marker element (112) may include frustoconical, spherical, egg-shaped, toroidal, and/or etc.

Marker element (112) includes a generally deformable and biocompatible material. As will be described in greater detail below, marker element (112) is generally configured to be manipulated into various predetermined shapes and then maintain such predetermined shapes over time. Thus, marker element (112) may include a material with at least some malleability. In other words, marker element (112) may be configured to be shaped by external forces above a predetermined threshold; yet be also configured to maintain a predetermined shape in response to external forces below a predetermined threshold. Examples of suitable materials may include, for example, stainless steel, platinum-chromium, cobalt-chromium, polymers, combinations thereof, and/or etc. In other versions, marker element (112) may include nitinol or one or more portions of nitinol. In such versions, the nitinol material may provide at least some shape memory properties where marker element (112) may change from one shape or configuration to another in response to heat or other stimulus from tissue. Regardless of the particular material used, in some versions, one or more surfaces of such materials may be etched with an echogenic pattern to enhance detection of marker element (112) under ultrasound.

As described above, marker element (112) includes a plurality of anchors (120) extending outwardly from body (114). Each anchor (120) is generally configured to engage tissue to increase the propensity of marker element (112) to remain in a fixed position within tissue. In the present version, each anchor (120) is formed as a generally straight section of wire extending from one or more legs (116). Although anchors (120) of the present version are generally straight, in other versions, anchors (120) may be curved or various alternative shapes. Additionally, although not shown, in some versions, anchors (120) may include barbs or other tissue engagement feature to further facilitate engagement of anchors (120) with tissue.

Each anchor (120) of the present version is generally at an angle relative to a longitudinal axis of marker element (112). The particular angle uses for each anchor (120) may vary with all anchors (120) or between individual anchors (120). For instance, in some versions, all anchors (120) may be oriented at an angle between about 30° and 60° relative to the longitudinal axis of marker element (112). In such versions, the particular angle used may be substantially the same for all anchors (120) (e.g., about) 45°. In other versions, each anchor (120) (or a set of anchors (120)) may have a different angle relative to other anchors (120).

Marker element (112) of the present version includes four anchors (120), although other suitable numbers of anchors (120) may be used in other versions. In the present configuration, anchors (120) are arranged at opposing ends of marker element (112). In other versions, various alternative anchor (120) configuration may be used depending on the particular number of anchors (120) used. For instance, in some versions, eight anchors (120) may be used with each anchor (120) being on an opposing segment of marker element (112). In other versions, two anchors (120) may be used with each anchor (120) being on an opposing end of marker element (112). In still other versions, anchors (120) may be omitted entirely.

### B. Example of Marker Delivery Device

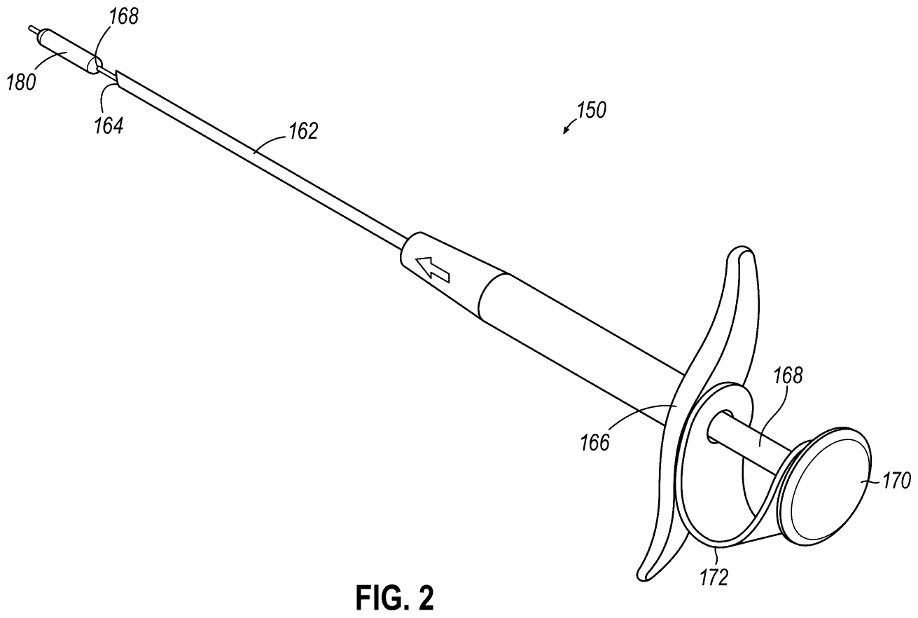

In some versions, it may be desirable to deploy marker (100) (or other markers described herein) within a body cavity using certain marker delivery devices. For instance, FIG. 2 shows an example marker delivery device (150) which includes an elongate outer cannula (162) having a marker exit, such as a distal opening (164) formed within the distal end of cannula (162). Thus, marker delivery device (150) of the present version is configured as an end deploy-style device. However, it should be understood that in other versions, a side deploy-style device may be used with a lateral opening disposed proximally of the distal end and on one side of cannula (162).

A grip (166) can be provided at the proximal end of cannula (162). A push rod (168) can be provided, with push rod (168) extending coaxially in cannula (162) such that push rod (168) is configured to translate within cannula (162) to displace one or more markers through distal opening (164). Push rod (168) may have sufficient rigidity in compression to push a marker from an internal lumen of cannula (162) out through distal opening (164) yet be relatively flexible in bending. A plunger (170) may be coupled at the proximal end of push rod (168) for forcing rod (168) distally in cannula (162) to deploy a marker out of cannula (162).

A user may grasp grip (166) with two fingers, and may push on plunger (170) using the thumb on the same hand, so that marker delivery device (150) is operated by a user's single hand. To facilitate single handed use, a spring (172) or other resilient feature may be provided about rod (168) between grip (166) and plunger (170) to bias rod (168) proximally relative to grip (166) and cannula (162).

Cannula (162) may be formed of any suitable metallic or non-metallic material. In some versions, cannula (162) is formed of a thin-walled hollow tube formed of a suitable medical grade plastic or polymer. One suitable material is a thermoplastic elastomer, such as Polyether block amide (PEBA), such as is known under the tradename PEBAX. Cannula (162) may be formed of PEBAX, and may be substantially transparent to visible light and X-ray.

Marker delivery device (150) of the present version further includes a deployment balloon (180) disposed on the distal end of push rod (168). It should be understood that deployment balloon (180) is merely optional and may be omitted in some versions. For instance, when marker delivery device (150) is used in combination with markers similar to marker (100) described above, deployment balloon (180) may be desirable to facilitate expansion of maker (100) from a pre-deployment to a post-deployment configuration as will be described in greater detail below. However, where marker delivery device (150) is used in combination with other markers that either do not require expansion or are self-expanding, deployment balloon (180) may be omitted and push rod (168) may be used to push such markers through distal opening (164).

In versions of marker delivery device (150) that include deployment balloon (180), deployment balloon (180) may be configured to exert a force on a marker similar to marker (100) described above to deform certain element of the marker. Thus, deployment balloon (180) of the present version may be configured as a non-compliant balloon to be of sufficent strength to withstand pressures associated with the application of force on elements such as marker (100).

Although not shown, it should be understood that deployment balloon (180) may be inflated by a variety of mechanisms. For instance, in some versions, push rod (168) may include a lumen configured to permit the injection of fluid into deployment balloon (180) via the lumen. Fluid injection in such examples may be performed by way of a variety of mechanisms such as syringes, fluid dispensing devices, and/or etc. In addition, in some versions, fluid injection may be linked to plunger (170) such that fluid injection may occur automatically with actuation of plunger (170). Various alternative mechanisms for expansion of deployment balloon (180) will be apparent to those of ordinary skill in the art in view of the teachings herein.

C. Illustrative Use of Biopsy Site Marker with Marker Delivery Device

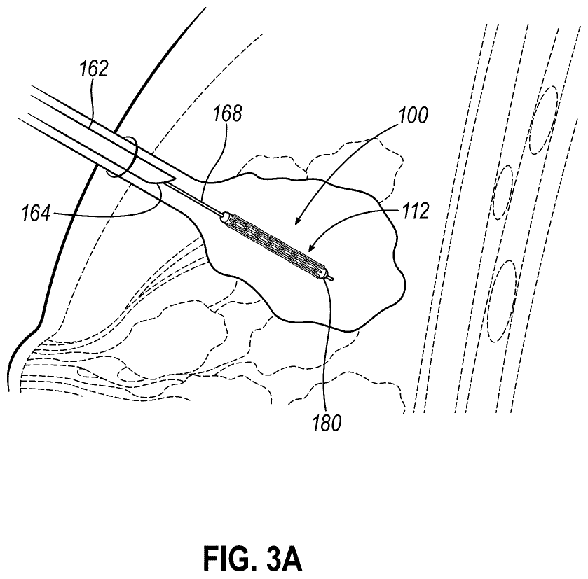

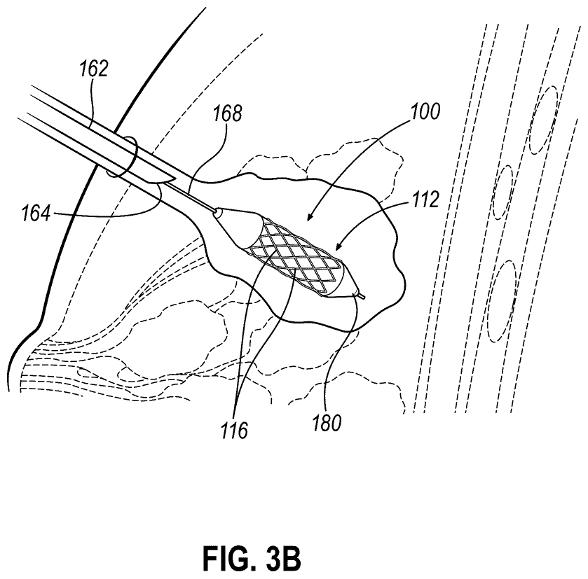

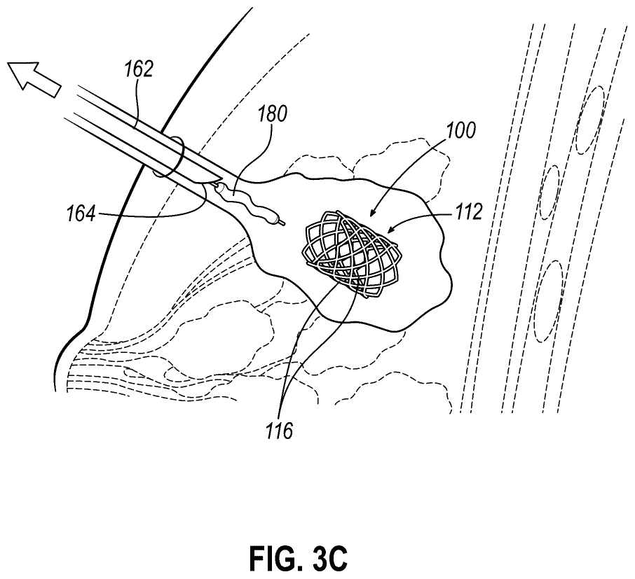

FIGS. 3A through 3C show an illustrative use of marker (100) in combination with marker delivery device (150) to deploy marker (100) within tissue. In the present use, marker (100) may be deployed at a biopsy site, although marker (100) may be deployed in a variety of tissue features in other uses. Prior to use, marker (100) may be inserted onto a deployment balloon (180) of marker delivery device (150). Both marker (100) and deployment balloon (180) may then be disposed within cannula (162) of marker delivery device (150) for initial insertion of cannula (162) into tissue.

In the present use, marker delivery device (150) may be first placed at the biopsy site. As can be seen in FIG. 3A, placement at the biopsy site may be performed by inserting cannula (162) through a tissue canal or passageway formed by a biopsy needle. Thus, marker delivery device (150) may be inserted after removal of a biopsy needle and/or other components of associated with biopsy needle such as targeting sleeves, introducers, and/or etc. In other uses, marker delivery device (150) may be used in combination with a biopsy needle, targeting sleeve, introducer, or other component. Use of such additional components may be desirable to maintain the tissue canal or passageway for ease of insertion of cannula (162). Use of such additional components may be further desirable to more readily identify the location of the biopsy site, thereby promoting accurate placement of marker (100) at the biopsy site.

Once cannula (162) of marker delivery device (150) is inserted into tissue, push rod (168) may be actuated using plunger (170). As best seen in FIG. 3A, such actuation may result push rod (170) extending the combination of deployment balloon (180) and marker (100) out of distal opening (164) of cannula (162) and into position at the biopsy site. At this point, the location of marker (100) may be confirmed using any suitable form of visualization such as ultrasound, x-ray, MRI, and/or etc.

Once marker (100) is positioned as desired, deployment balloon (180) may be used to expand marker element (112) from a pre-deployment configuration shown in FIG. 3A to a post-deployment configuration shown in FIGS. 3B and 3C. As can be best seen in FIG. 3B, deployment balloon (180) may be inflated or otherwise expanded to exert a force on marker element (112). This force may likewise expand marker element (112) by deforming the shape formed by legs (116). Expansion may continue until one or more legs (116) and/or anchors (120) engage the surrounding tissue, thereby holding marker (100) in a desired position within tissue.

Once deployment balloon (180) is expanded to manipulate marker element (112) into the post-deployment configuration, deployment balloon (180) may be deflated. Marker delivery device (150) may be removed from the tissue as shown in FIG. 3C, leaving marker (100) in place at the biopsy site. Due to the configuration of marker element (112) described above, marker element (112) may remain in the post-deployment configuration even after removal of deployment balloon (180). Marker element (112) may maintain this configuration over time, thereby maintaining the position of marker (100) at the biopsy site for subsequent follow-up procedures.

### II. EXAMPLE BIOPSY SITE MARKER WITH EXPANDABLE CORE

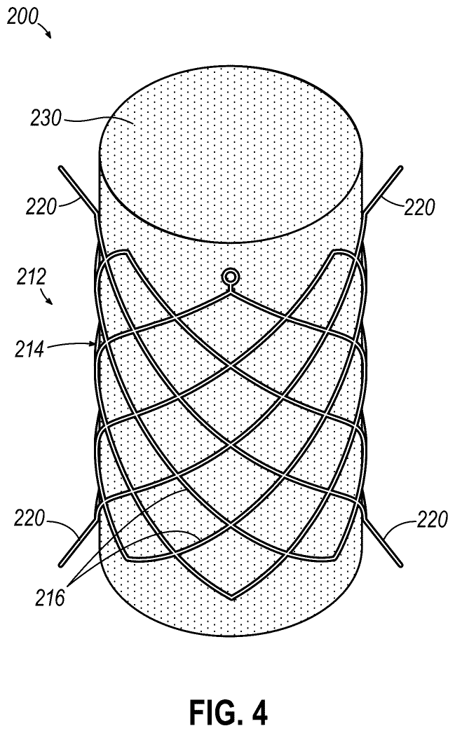

FIG. 4 shows an example of an alternative marker (200) that is similar to marker (100) described above. For instance, like marker (100) described above, marker (200) is generally configured to expand from a pre-deployment configuration to a post-deployment configuration to thereby anchor or otherwise fix marker (200) within tissue at a predetermined location. However, unlike marker (100) described above, marker (200) of the present version is generally self-expandable such that an external structure such as deployment balloon (180) may not be needed to initiate the transition from the pre-deployment configuration to the post-deployment configuration. In some circumstances, the self-expansion mechanism described herein may be desirable to permit use of marker (200) with a variety of deployment devices with or without structures similar to deployment balloon (180). As will be described in greater detail below, the self-expansion mechanism described herein may be desirable to enhance visualization of marker (200) in certain circumstances.

As with marker (100) described above, marker (200) of the present version includes a marker element (212). Marker element (212) of the present version is substantially similar to marker element (112) described above. For instance, as similarly described above, marker element (212) may be generally configured as non-bioabsorbable and radiopaque and/or echogenic to enhance visualization using one or more modes of visualization.

As with marker element (112) described above, marker element (212) of the present version includes a body (214) and a plurality of anchors (220) extending outwardly from one or more portions of body (214). Body (214) is substantially similar to body (114) described above. For instance, body (214) is generally configured to readily expand and contract between different positions, as will be described in greater detail below. As with body (114) described above, body (214) in the present version includes a plurality of interconnecting legs (216) that may form a variety of repeating shapes and/or patterns.

As with legs (116) described above, legs (216) are generally configured to facilitate deformation of marker element (212) between one or more different positions. Thus, the particular shape formed by legs (216) is generally configured permit at least some movement of individual legs (216). Some flexion of individual legs (216) relative to other legs (216) may be desirable to permit deformation of marker element (212) generally and/or one or more portions of marker element (212). The particular shape formed by legs (216) may also be desirable to provide marker element (212) with a relatively large footprint and/or surface area using a relatively small volume of material. The particular shape formed by legs (216) may additionally be desirable to provide openings within the surface of marker element (212) to facilitate tissue in growth, thereby providing additional tissue anchoring and/or fixation over time.

As with legs (116) described above, legs (216) of the present version are interconnected with each other to form a general shape of marker element (212). As similarly described above, marker element (212) is formed in the shape of a hollow cylinder (e.g., stent-shaped, either straight or tapered). Although both ends of marker element (212) are open in the present version, in other versions, one or both ends may be closed. In still other versions, a variety of alternative shapes may be used. Suitable alternative shapes for marker element (212) may include frustoconical, spherical, egg-shaped, toroidal, and/or etc.

Marker element (212) also includes a plurality of anchors (220) substantially similar to anchors (120) described above. As with anchors (120), anchors (220) of the present version extend outwardly from body (214) and are generally configured to engage tissue to increase the propensity of marker element (212) to remain in a fixed position within tissue. Each anchor (220) is formed as a generally straight section of wire extending from one or more legs (216). Although anchors (220) of the present version are generally straight, in other versions, anchors (220) may be curved or various alternative shapes. Additionally, although not shown, in some versions, anchors (220) may include barbs or other tissue engagement feature to further facilitate engagement of anchors (220) with tissue.

Like each anchor (120) described above, each anchor (220) of the present version is generally at an angle relative to a longitudinal axis of marker element (212). As similarly described above, the particular angle used for each anchor (220) may vary with all anchors (220) or between individual anchors (220). Additionally, although marker element (212) of the present version includes four anchors (220), other suitable numbers of anchors (220) may be used.

Unlike marker (100) described above, marker (200) of the present version includes a carrier (230) in addition to marker element (212). Carrier (230) is generally configured to expand within tissue to exert a force on marker element (212), thereby transitioning marker element (212) from the pre-deployment configuration to the post-deployment configuration. Additionally, carrier (230) may be generally configured to enhance the visibility of marker (200) under certain visualization modalities such as ultrasonic visualization.

Carrier (230) of the present version includes a bioabsorbable material that may be configured absorb into tissue after placement of marker (200) at a biopsy site. The material of carrier (230) is further configured to expand or swell after placement of marker (200) at a biopsy site. In some versions, the material of carrier (230) may be configured to swell two to three times or more relative to an initial size. As will be described in greater detail below, these properties may be desirable to permit carrier (230) to drive movement of marker element (212) from the pre-deployment configuration to the post-deployment configuration. Examples of suitable materials for carrier (230) may include collagen, hydrogel, combinations of collagen and hydrogel, and/or etc. Although not shown, it should be understood that in some versions, the material of carrier (230) may include bubbles and/or microbubbles to promote echogenicity of carrier (230) under ultrasound visualization.

Carrier (230) defines a generally cylindrical shape. As will be described in greater detail below, the particular shape of carrier (230) generally corresponds to the shape of marker element (212). Thus, in versions where the shape of marker element (212) is varied, the shape of carrier (230) may likewise be varied. Additionally, the axial length of carrier (230) may be greater than the axial length of marker element (212) so that one or more portions of carrier (230) may protrude from marker element (212). In other versions, the axial length of carrier (230) may be less than or equal to the axial length of marker element (212).

Carrier (230) is positioned within marker element (212) such that marker element (212) wraps around an outer surface of carrier (230). Thus, carrier (230) is positioned relative to marker element (212) to act as a core of marker element (212). The particular diameter of carrier (230) may be approximately equal to the inner diameter of marker element (212) when marker element (212) is in the pre-deployment configuration. In some versions, there may be a slight interference between the outer surface of carrier (230) and the inner diameter of marker element (212). Such an interference may be desirable to secure marker element (212) onto carrier (230), which may promote ease of use by keeping marker element (212) and carrier (230) secured together as a discrete unit. In still other versions, one or more portions of marker element (212) may be embedded within carrier (230) to further facilitate additional coupling of marker element (212) to carrier (230).

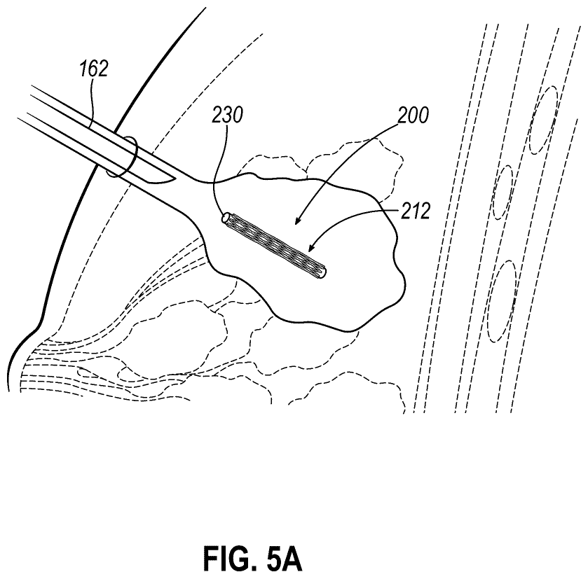

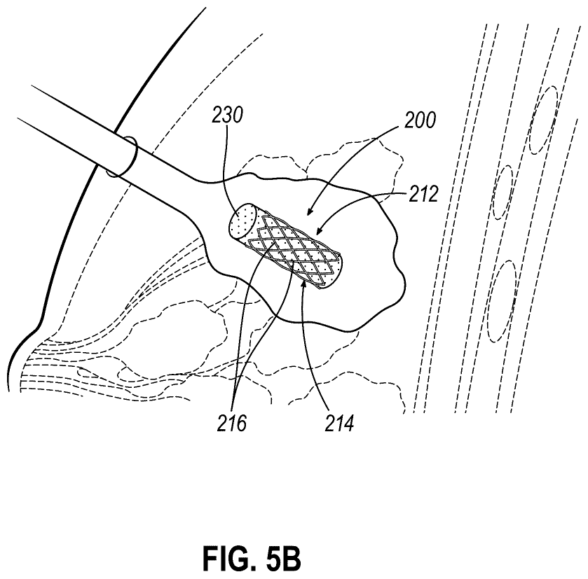

FIGS. 5A through 5B show an illustrative use of marker (200) described above to deploy marker (200) within tissue. In the present use, marker (200) may be deployed at a biopsy site, although marker (200) may be deployed in a variety of tissue features in other uses. Prior to use, marker (200) may be inserted onto a marker delivery device similar to marker delivery device (150) described above. Unlike the use with respect to marker (100) described above, structures similar to deployment balloon (180) may be unnecessary. Thus, marker (200) may be inserted directly into a cannula similar to cannula (166) described above. A structure similar to push rod (168) may then be used to manipulate marker (200) directly (168) in lieu of deployment balloon (180).

After insertion into a marker delivery device similar to marker delivery device (150), the marker delivery device may be manipulated into position at the biopsy site. Placement at the biopsy site may be performed by inserting a structure similar to cannula (162) through a tissue canal or passageway formed by a biopsy needle. Thus, insertion may occur after removal of a biopsy needle and/or other components of associated with biopsy needle such as targeting sleeves, introducers, and/or etc. In other uses, devices such as marker delivery device (150) may be used in combination with a biopsy needle, targeting sleeve, introducer, or other component. Use of such additional components may be desirable to maintain the tissue canal or passageway for ease of insertion. Use of such additional components may be further desirable to more readily identify the location of the biopsy site, thereby promoting accurate placement of marker (200) at the biopsy site.

Once a structure similar to cannula (162) of marker delivery device (150) is inserted into tissue, push rod (168) or other similar structure may be actuated using plunger (170) or other actuation mechanisms. Marker (200) may then be manipulated out of the structure similar to cannula (162) and into the biopsy site into the position shown in FIG. 5A. At this point, the location of marker (200) may be confirmed using any suitable form of visualization such as ultrasound, x-ray, MRI, and/or etc.

Once marker (200) is positioned as desired, carrier (230) may swell or otherwise expand by absorbing fluid at the biopsy site. As carrier (230) expands, carrier (230) may drive corresponding expansion of marker element (212) from a pre-deployment configuration shown in FIG. 5A to a post-deployment configuration shown in FIG. 5B. As can be best seen in FIG. 5B, expansion to the post-deployment configuration may expand marker element (212) by deforming the shape formed by legs (216). Expansion may continue until one or more legs (216) and/or anchors (220) engage the surrounding tissue, thereby holding marker (200) in a desired position within tissue.

Once carrier (230) is expanded to manipulate marker element (212) into the post-deployment configuration, carrier (230) may then begin to degrade and/or bioabsorb. Thus, carrier (230) may disappear over time with only marker element (212) remaining at the biopsy site. It should be understood that the particular material of marker element (212) may be rigid enough to maintain the post-deployment configuration even after absorption of carrier (230), yet be flexible enough to permit manipulation of marker element (212) by carrier (230). Marker element (212) may maintain this configuration over time, thereby maintaining the position of marker (200) at the biopsy site for subsequent follow-up procedures.

### III. EXAMPLE BIOPSY SITE MARKER WITH SELF-EXPANDING MESH

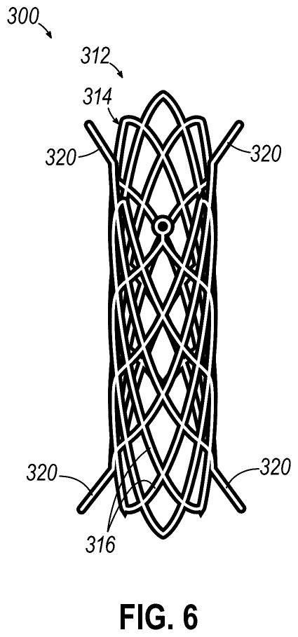

FIG. 6 shows an example of an alternative marker (300) that is similar to marker (100) described above. For instance, like marker (100) described above, marker (300) is generally configured to expand from a pre-deployment configuration to a post-deployment configuration to thereby anchor or otherwise fix marker (300) within tissue at a predetermined location. However, unlike marker (100) described above, marker (300) of the present version is generally self-expandable such that an external structure such as deployment balloon (180) may not be needed to initiate the transition from the pre-deployment configuration to the post-deployment configuration. In some circumstances, the self-expansion mechanism described herein may be desirable to permit use of marker (300) with a variety of deployment devices with or without structures similar to deployment balloon (180). As will be described in greater detail below, the self-expansion mechanism described herein may be desirable to enhance visualization of marker (300) in certain circumstances.

As with marker (100) described above, marker (300) of the present version includes a marker element (312). Marker element (312) of the present version is substantially similar to marker element (112) described above, except where otherwise noted herein. For instance, as similarly described above, marker element (312) may be generally configured as radiopaque and/or echogenic to enhance visualization using one or more modes of visualization.

As with marker element (112) described above, marker element (312) of the present version includes a body (314) and a plurality of anchors (320) extending outwardly from one or more portions of body (314). Body (314) is substantially similar to body (114) described above. For instance, body (314) is generally configured to readily expand and contract between different positions, as will be described in greater detail below. As with body (114) described above, body (314) in the present version includes a plurality of interconnecting legs (316) that may form a variety of repeating shapes and/or patterns.

As with legs (116) described above, legs (316) are generally configured to facilitate deformation of marker element (312) between one or more different positions. Thus, the particular shape formed by legs (316) is generally configured permit at least some movement of individual legs (316). Some flexion of individual legs (316) relative to other legs (316) may be desirable to permit deformation of marker element (312) generally and/or one or more portions of marker element (312). The particular shape formed by legs (316) may also be desirable to provide marker element (312) with a relatively large footprint and/or surface area using a relatively small volume of material. The particular shape formed by legs (316) may additionally be desirable to provide openings within the surface of marker element (312) to facilitate tissue in growth, thereby providing additional tissue anchoring and/or fixation over time.

As with legs (116) described above, legs (316) of the present version are interconnected with each other to form a general shape of marker element (312). As similarly described above, marker element (312) is formed in the shape of a hollow cylinder (e.g., stent-shaped, either straight or tapered). Although both ends of marker element (312) are open in the present version, in other versions, one or both ends may be closed. In still other versions, a variety of alternative shapes may be used. Suitable alternative shapes for marker element (312) may include frustoconical, spherical, egg-shaped, toroidal, and/or etc.

Marker element (312) also includes a plurality of anchors (320) substantially similar to anchors (120) described above. As with anchors (120), anchors (320) of the present version extend outwardly from body (314) and are generally configured to engage tissue to increase the propensity of marker element (312) to remain in a fixed position within tissue. Each anchor (320) is formed as a generally straight section of wire extending from one or more legs (316). Although anchors (320) of the present version are generally straight, in other versions, anchors (320) may be curved or various alternative shapes. Additionally, although not shown, in some versions, anchors (320) may include barbs or other tissue engagement feature to further facilitate engagement of anchors (320) with tissue.

Like each anchor (120) described above, each anchor (320) of the present version is generally at an angle relative to a longitudinal axis of marker element (312). As similarly described above, the particular angle used for each anchor (320) may vary with all anchors (320) or between individual anchors (320). Additionally, although marker element (312) of the present version includes four anchors (320), other suitable numbers of anchors (320) may be used.

Unlike marker (100) described above, marker (300) of the present version includes a self-deformable material. In particular, marker element (312) includes a bio-compatible material configured to transform marker element (312) from the pre-deployment configuration to the post-deployment configuration upon deployment of marker (300) at a biopsy site. By way of example only, suitable self-deformable materials may include hydrogel, collagen, various combinations of hydrogel and collagen, and/or etc. Hydrogel and collagen may be desirable materials due to material properties involving swelling and/or expansion when exposed to moisture from tissue. Hydrogel and collagen may additionally be desirable to improve echogenicity of marker element (312).

Although marker element (312) may be comprised entirely of hydrogel and/or collagen, other materials may be used in combination with hydrogel and/or collagen in some versions. For instance, in some versions, marker element (312) may include a hydrogel and/or collagen exterior coating with a metallic wire or polymer core disposed within the coating. In such versions, the hydrogel and/or collagen coating may provide the self-expanding and echogenic properties described above. Meanwhile, the metallic wire or polymer core may provide enhanced rigidity to marker element (312). Additionally, hydrogel and/or collagen materials are generally bioabsorbable. Thus, a metallic wire or polymer core may be desirable to provide a permanent structure for visualization of marker (300) after absorption of hydrogel and/or collagen materials into tissue.

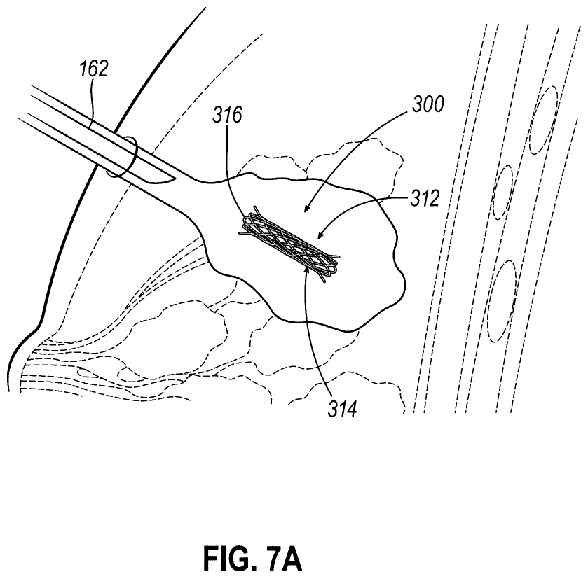

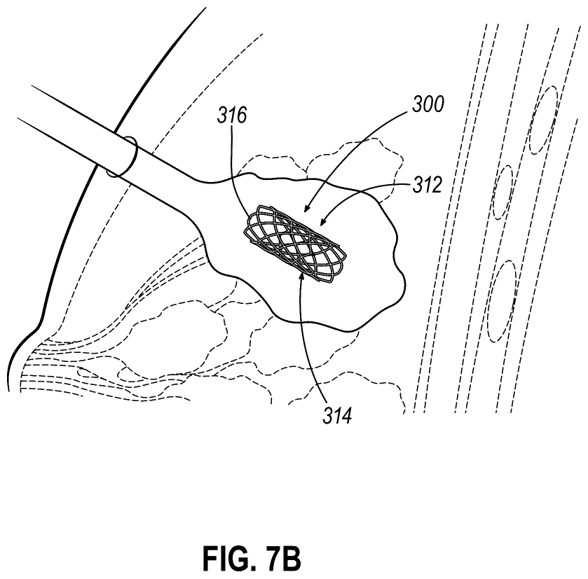

FIGS. 7A and 7B show an illustrative use of marker (300) described above to deploy marker (300) within tissue. In the present use, marker (300) may be deployed at a biopsy site, although marker (300) may be deployed in a variety of tissue features in other uses. Prior to use, marker (300) may be inserted onto a marker delivery device similar to marker delivery device (150) described above. Unlike the use with respect to marker (100) described above, structures similar to deployment balloon (180) may be unnecessary. Thus, marker (300) may be inserted directly into a cannula similar to cannula (166) described above. A structure similar to push rod (168) may then be used to manipulate marker (300) directly (168) in lieu of deployment balloon (180).

After insertion into a marker delivery device similar to marker delivery device (150), the marker delivery device may be manipulated into position at the biopsy site. Placement at the biopsy site may be performed by inserting a structure similar to cannula (162) through a tissue canal or passageway formed by a biopsy needle. Thus, insertion may occur after removal of a biopsy needle and/or other components of associated with biopsy needle such as targeting sleeves, introducers, and/or etc. In other uses, devices such as marker delivery device (150) may be used in combination with a biopsy needle, targeting sleeve, introducer, or other component. Use of such additional components may be desirable to maintain the tissue canal or passageway for ease of insertion. Use of such additional components may be further desirable to more readily identify the location of the biopsy site, thereby promoting accurate placement of marker (300) at the biopsy site.

Once a structure similar to cannula (162) of marker delivery device (150) is inserted into tissue, push rod (168) or other similar structure may be actuated using plunger (170) or other actuation mechanisms. Marker (300) may then be manipulated out of the structure similar to cannula (162) and into the biopsy site into the position shown in FIG. 7A. At this point, the location of marker (300) may be confirmed using any suitable form of visualization such as ultrasound, x-ray, MRI, and/or etc.

Once marker (300) is positioned as desired, the one or more materials forming marker element (312) may swell or otherwise expand by absorbing fluid at the biopsy site. As the materials forming marker element (312) expand, corresponding expansion of marker element (312) may occur. In particular, marker element (312) may initially be in the pre-deployment configuration shown in FIGS. 6 and 7A. In the pre-deployment configuration, the hydrogel and/or collagen forming marker element (312) may be in a dehydrated and/or compressed configuration. This dehydrated and/or compressed configuration may permit expansion of marker element (312) to the post-deployment configuration shown in FIG. 7B. As can be seen, expansion to the post-deployment configuration may expand marker element (312) by deforming the shape formed by legs (316). Expansion may continue until one or more legs (316) and/or anchors (320) engage the surrounding tissue, thereby holding marker (300) in a desired position within tissue.

Once the materials of marker element (312) are expanded to manipulate marker element (312) into the post-deployment configuration, such materials may then begin to degrade and/or absorb. Thus, at least some of marker element (312) may disappear over time with only a non-bioabsorbable portion of marker element (312) remaining at the biopsy site. It should be understood that the particular non-bioabsorbable material portion of marker element (312) may be rigid enough to maintain the post-deployment configuration even after absorption of bioabsorbable portions; yet be flexible enough to permit manipulation of marker element (312) by the bioabsorbable portions. Marker element (312) may maintain this configuration over time, thereby maintaining the position of marker (300) at the biopsy site for subsequent follow-up procedures.

### IV. EXAMPLE COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A biopsy site marker, comprising: a carrier, the carrier being configured to expand in diameter in response to moisture; and a marker element, the marker element including a base portion, the base portion defining a plurality of interconnected legs, each leg being configured to move relative to another leg to permit movement of the marker element from a pre-deployment configuration to a post-deployment configuration, the base portion being disposed around an outer surface of the carrier, the carrier being configured to drive movement of the marker element from the pre-deployment configuration to the post-deployment configuration.

### Example 2

The marker of Example 1, the outer surface of the carrier defining a first diameter, the base portion defining a second diameter, the first diameter corresponding to the second diameter.

### Example 3

The marker of Example 1, the outer surface of the carrier defining a first diameter, the base portion defining a second diameter, the second diameter being less than the first diameter to provide an interference fit between the carrier and the base portion.

### Example 4

The marker of any of Examples 2 and 3, the carrier being configured to expand the second diameter defined by the base portion when driving movement of the marker element from the pre-deployment configuration to the post-deployment position.

### Example 5

The marker of any of Examples 2 and 3, the carrier being configured to expand the second diameter defined by the base portion by three times or more when driving movement of the marker element from the pre-deployment configuration to the post-deployment position.

### Example 6

The marker of any of Examples 1 through 5, the marker element including a material, the material having at least some rigidity such that the base portion is configured to maintain a predetermined shape in absence of the carrier.

### Example 7

The marker of any of Examples 1 through 6, the carrier being bioabsorbable, the marker element being non-bioabsorbable.

### Example 8

The marker of any of Examples 1 through 7, the carrier being configured to deform a shape defined by the plurality of legs during expansion of the carrier.

### Example 9

The marker of any of Examples 1 through 8, the carrier defining a cylindrical shape, the body portion of the marker element defining a shape corresponding to the carrier.

### Example 10

The marker of any one or more of Examples 1 through 9, the carrier defining a first axial length, the body portion of the marker element defining a second axial length, the first axial length being longer than the second axial length.

### Example 11

The marker of any of Examples 1 through 10, the base portion further including one or more anchors, each anchor of the one or more anchors extending outwardly from one or more legs of the plurality of legs.

### Example 12

The marker of any of Examples 1 through 10, the base portion further including one or more anchors, each anchor of the one or more anchors extending outwardly from one or more legs of the plurality of legs, each anchor of the one or more anchors being oriented at an angle relative to a longitudinal axis defined by the base portion.

### Example 13

The marker of any of Examples 1 through 12, the marker element including a surface treatment to one or more exterior surfaces of the marker element, the surface treatment being configured to increase echogenicity of the marker element.

### Example 14

The marker of any of Examples 1 through 13, the carrier including a collagen material.

### Example 15

The marker of any of Examples 1 through 14, the carrier including a hydrogel material.

### Example 16

A biopsy site marker, comprising: a marker element, the marker element including a base portion and an anchor portion, the anchor portion extending from the base portion, the base portion defining a plurality of interconnected legs, each leg being configured to move relative to another leg to transition the marker element from a pre-deployment configuration to a post-deployment configuration, the anchor being configured to engage tissue when the marker element is in the post-deployment configuration.

### Example 17

The biopsy site marker of Example 16, the plurality of interconnected legs together defining a mesh structure.

### Example 18

The biopsy site marker of any of Examples 16 or 17, the plurality of interconnected legs together defining a plurality of diamond-shaped openings extending through the base portion.

### Example 19

The biopsy site marker of any of Examples 16 through 18, the marker element defining a hollow interior, the hollow interior being configured to receive a deployment balloon.

### Example 20

The biopsy site marker of any of Examples 16 through 19, the marker element including a self-deforming material, the self-deforming material being configured transition the marker element from the pre-deployment configuration to the post-deployment configuration upon contact with tissue at a biopsy site.

### Example 21

The biopsy site marker of any of Examples 16 through 19, the marker element including a self-deforming material, the self-deforming material being configured transition the marker element from the pre-deployment configuration to the post-deployment configuration upon contact with tissue at a biopsy site, the self-deforming material defining each leg of the plurality of interconnected legs.

### Example 22

The biopsy site marker of any of Examples 16 through 21, further comprising a carrier, the carrier being coupled to a portion of the marker element, the carrier being bioabsorbable.

### Example 23

The biopsy site marker of any of Examples 16 through 21, further comprising a carrier, the carrier being coupled to a portion of the marker element, the carrier being bioabsorbable, the body portion of the marker element being wrapped around a portion of the carrier.

### Example 24

The biopsy site marker of any of Examples 16 through 21, further comprising a carrier, the carrier being coupled to a portion of the marker element, the carrier being bioabsorbable, a portion of the marker element being embedded within a portion of the carrier.

### Example 25

A method for securing a biopsy site marker at a biopsy site, the method comprising: inserting a marker delivery device into tissue to position a deployment opening of a cannula at a biopsy site; actuating a push rod to extend a delivery balloon from the deployment opening of the cannula into the biopsy site, the delivery balloon having a marker element disposed on a portion thereof; expanding the delivery balloon to deform the marker element; and removing the delivery balloon from the biopsy site while the marker element remains in position at the biopsy site.

### Example 26

The method of Example 25, the step of expanding the delivery balloon including inflating the delivery balloon.

### Example 27

The method of any of Examples 25 or 26, the step of expanding the delivery balloon including expanding the delivery balloon to deform the marker element until the marker element contacts at least some tissue at the biopsy site.

### Example 28

The method of any of Examples 25 through 27, the act of deforming the marker element including moving a plurality of interconnected legs relative to each other to increase a volume of space occupied by the marker element without increasing a volume of material defined by the marker element.

### Example 29

The method of any of Examples 25 through 28, the step of expanding the delivery balloon including moving one or more anchors extending from a portion of the marker element into engagement with at least some tissue at the biopsy site.

### Example 30

A biopsy marking system, the system comprising: a biopsy site marker of any one or more of Examples 1 through 24; a marker delivery device, the marker delivery device including an introducer cannula, a grip, and a push rod, the introducer cannula being configured to receive the marker, the push rod being configured to move relative to the grip to eject the marker from a marker exit defined by the introducer cannula.

### Example 31

The biopsy marking system of Example 30, the marker delivery device further including a deployment balloon, the deployment balloon being configured to receive a portion of the biopsy site marker.

### V. CONCLUSION

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A biopsy site marker, comprising:
a carrier, the carrier being configured to expand in diameter in response to moisture; and
a marker element, the marker element including a base portion,
the base portion defining a plurality of interconnected legs, each leg being configured to move relative to another leg to permit movement of the marker element from a pre-deployment configuration to a post-deployment configuration,
the base portion being disposed around an outer surface of the carrier, the carrier being configured to drive movement of the marker element from the pre-deployment configuration to the post-deployment configuration.

2. The marker of claim 1, the outer surface of the carrier defining a first diameter, the base portion defining a second diameter, the first diameter corresponding to the second diameter.

3. The marker of claim 1, the outer surface of the carrier defining a first diameter, the base portion defining a second diameter, the second diameter being less than the first diameter to provide an interference fit between the carrier and the base portion.

4. The marker of claim 2, the carrier being configured to expand the second diameter defined by the base portion when driving movement of the marker element from the pre-deployment configuration to the post-deployment position.

5. The marker of claim 2, the carrier being configured to expand the second diameter defined by the base portion by three times or more when driving movement of the marker element from the pre-deployment configuration to the post-deployment position.

6. The marker of claim 1, the marker element including a material, the material having at least some rigidity such that the base portion is configured to maintain a predetermined shape in absence of the carrier.

7. The marker of claim 1, the carrier being bioabsorbable, the marker element being non-bioabsorbable.

8. The marker of claim 1, the carrier being configured to deform a shape defined by the plurality of legs during expansion of the carrier.

9. The marker of claim 1, the carrier defining a cylindrical shape, the body portion of the marker element defining a shape corresponding to the carrier.

10. The marker of claim 1, the carrier defining a first axial length, the body portion of the marker element defining a second axial length, the first axial length being longer than the second axial length.

11. The marker of claim 1, the base portion further including one or more anchors, each anchor of the one or more anchors extending outwardly from one or more legs of the plurality of legs.

12. The marker of claim 1, the base portion further including one or more anchors, each anchor of the one or more anchors extending outwardly from one or more legs of the plurality of legs, each anchor of the one or more anchors being oriented at an angle relative to a longitudinal axis defined by the base portion.

13. The marker of claim 1, the marker element including a surface treatment to one or more exterior surfaces of the marker element, the surface treatment being configured to increase echogenicity of the marker element.

14. The marker of claim 1, the carrier including a collagen material or a hydrogel material.

15. (canceled)

16. A biopsy site marker, comprising:
a marker element, the marker element including a base portion and an anchor portion,
the anchor portion extending from the base portion,
the base portion defining a plurality of interconnected legs, each leg being configured to move relative to another leg to transition the marker element from a pre-deployment configuration to a post-deployment configuration,
the anchor being configured to engage tissue when the marker element is in the post-deployment configuration.

17. The biopsy site marker of claim 16, the plurality of interconnected legs together defining a mesh structure.

18. The biopsy site marker of claim 16, the plurality of interconnected legs together defining a plurality of diamond-shaped openings extending through the base portion.

19. The biopsy site marker of claim 16, the marker element defining a hollow interior, the hollow interior being configured to receive a deployment balloon.

20. The biopsy site marker of claim 16, the marker element including a self-deforming material, the self-deforming material being configured transition the marker element from the pre-deployment configuration to the post-deployment configuration upon contact with tissue at a biopsy site.

21.-24. (canceled)

25. A method for securing a biopsy site marker at a biopsy site, the method comprising:
(a) inserting a marker delivery device into tissue to position a deployment opening of a cannula at a biopsy site;
(b) actuating a push rod to extend a delivery balloon from the deployment opening of the cannula into the biopsy site, the delivery balloon having a marker element disposed on a portion thereof;
(c) expanding the delivery balloon to deform the marker element; and
(d) removing the delivery balloon from the biopsy site while the marker element remains in position at the biopsy site.

26.-31. (canceled)
